(19) United States
(12) Patent Application Publication
(10) Pub. No.: US 2026/0294501 A1
(43) Pub. Date: Oct. 1, 2026

(54) DIRECTIONAL ABLATION DEVICE

(71) Applicant: CORFIGO, INC., Montclair, NJ (US)

(72) Inventor: Jason Sperling, Upper Saddle River, NJ (US)

(21) Appl. No.: 19/477,532

(22) PCT Filed: Apr. 24, 2024
(86) PCT No.: PCT/US2024/025968

§ 371 Date: Oct. 22, 2025

Related U.S. Application Data
(60) Provisional application No. 63/497,788, filed on Apr. 24, 2023.
(60) Provisional application No. 63/582,874, filed on Sep. 15, 2023.

(51) Int. Cl. A61B 18/02; A61B 17/00; A61B 18/00; A61B 90/00

(52) U.S. Cl. CPC A61B 18/02; A61B 90/04; A61B 90/06; A61B 2017/00557; A61B 2017/00561; A61B 2018/00184; A61B 2018/00214; A61B 2018/00351; A61B 2018/00577; A61B 2018/00791; A61B 2018/00875; A61B 2018/00892; A61B 2018/0212; A61B 2018/0262; A61B 2090/0436; A61B 2090/0463; A61B 2090/065

(57) ABSTRACT

Various ablation devices having an elongate body, an omnidirectional ablation element associated with the elongate body, and an ablation shielding structure attached to at least a portion of the omnidirectional ablation element, the ablation shielding structure comprising a low energy conductivity structure, wherein the ablation shielding structure is configured to resist energy conduction only on the first side of the omnidirectional ablation element, thereby increasing ablation potency on a second side opposite the first side.

### CROSS-REFERENCE TO RELATED APPLICATION(S)

This application claims priority to International PCT Application No. PCT/US2024/025968, filed on Apr. 24, 2024, which claims the benefit under 35 U.S.C. § 119(e) to U.S. Provisional Application 63/582,874, filed Sep. 15, 2023 and entitled “Directional Ablation Device,” and to U.S. Provisional Application 63/497,788, filed Apr. 24, 2023 and entitled “Directional Ablation Device,” both of which are hereby incorporated herein by reference in their entireties.

### FIELD

The various embodiments herein relate to medical devices that utilize ablation to treat diseased tissues, and especially to focal ablation devices for ablating challenging tissues.

### BACKGROUND

The use of different types of energy sources (heat, cooling, pulsed energy field, etc.) to treat diseased tissues (associated with conditions like heart rhythm disorders and malignant tumors, for example) has been long-established. While cryoablation technologies is the main focus of this section, most of the discussion herein also applies to the other forms of intentional tissue destruction (including those mentioned above) as well. Most treatment tools utilize cryogenic fluid (gas or liquid) such as argon, nitrous oxide or liquid nitrogen. One disadvantage of known ablation systems and commonly-used cryogenic energy sources is that they are ineffective for ablating certain target tissues. More specifically, they are ineffective for the ablation of thick tissues (such as left ventricle muscle) and tissue that is warmed by an intimately associated physiologic “warmer” (such as the blood pool flowing in the cardiac chambers).

Thus, for ablation of the most demanding tissues discussed above, the optimal cryogenic fluids are those having lower boiling points than nitrogen, such as neon, helium and hydrogen. More specifically, the low boiling point cryogenic fluids (also referred to herein as “extremely low temperature” or “XLT” cryogenic fluids) are those fluids with a boiling point that is lower than about 77 Kelvin (“K”).

Certain challenging tissue ablation targets require a very small, or ‘focal’ ablation footprint. For example, ventricular tachycardia (“VT”) can be treated via ablation of the left ventricle, which is responsible for producing significant cardiac output both at rest and with exercise. As such, it is critical to minimize damage to the left ventricle tissue. As a result, left ventricle ablation requires precisely targeted ablation in the smallest effective ablation volumes in order to minimize damage to the left ventricle tissue and thereby allow the left ventricle to continue to operate as the major workhorse cardiac chamber.

However, known focal ablation catheters have significant disadvantages. For example, such known catheters cannot penetrate the tissue thickness of the left ventricle with enough energy (or energy absorption, as in cryoablation) to be effective, especially given that the heart is an incredibly potent heater as mentioned above. Further, such catheters come with significant risk of collateral damage (unintended ablation of adjacent tissues). More specifically, ablation from inside the heart using such devices may be too potent in thinner tissues (such as the atrium, which is only 2-3 mm thick), and thus may cause damage to structures beyond the atrial wall (like the esophagus). Similarly, from outside of the heart or another organ, contact between the ablation element and non-target warm tissues will also decrease effectiveness of target ablation by absorbing heat from these additional sources unintentionally.

One example of a typical known cryoablation catheter device 1 is depicted in FIGS. 1A-1D. The device 1 has a cryofluid inlet line 3 and a cryofluid outlet line 4 that extend through the device 1 and into the ablation element 2 such that cold cryofluid is conducted via the inlet line 3 into the ablation element 2 and then flows out via cryofluid outlet line 4.

The unintentional (and undesirable) absorption of energy from adjacent, non-target tissues by such a device as described above is best shown in FIG. 1B, which depicts the cryoablation element 2 absorbing energy/heat from every possible direction, as represented by arrows 9. In some versions as shown in FIGS. 1C and 1D, these known devices can have a slidable shield or sheath 11. The shield 11 can be moved between a retracted position (as shown in FIG. 1C) in which the entire ablation element 2 is exposed and an extended position (as shown in FIG. 1D) in which some or all of the ablation element 2 is covered. The disadvantage of this shielded configuration is that while it can restrict some or all of the ablative energy of the ablation element 2, but when exposed, the ablation element 2 still provides ablative energy in an omnidirectional fashion as shown above.

There is a need in the art for an improved focal ablation device and related systems and methods.

### BRIEF SUMMARY

Certain embodiments are directed specifically at focal cryoablation devices that are intended for epicardial (external) ablation of the ventricle or other relevant cardiac structures and solid (non-cardiac) organs. Epicardial ablation means that the intention is to ablate a portion of the outer surface of the heart, and to not absorb energy from structures that are immediately adjacent or opposite the cardiac target. An important nuance of epicardial ventricular ablation, is that the space within which to operate and navigate is limited. The heart is positioned within a sac called the pericardium, and the space between the heart surface and the inside of the heart sac is minimal. The two surfaces (like flexible sheets) essentially touch one another but are separated by a thin layer of lubricating fluid (‘pericardial fluid’). The space has a limited degree of flexibility and pliability, such that a therapeutic device may be inserted into this space via a remote puncture location (usually below the breastbone), and navigated to a target location for therapy, such as ablation. A major limitation of using an ablation device in such a fashion is that typical ablation devices are omnidirectional—the ablation energy is effective in its entire available surface area. Without mitigation, such a 3-dimensional device, when navigated within the otherwise closed and intact pericardial sac, will contact not only the heart, but the inner wall of the pericardial sac itself. While the heart surface can be ablated, so will the pericardium sac in that location, and anything in proximity to, or attached to the sac. Structures immediately adjacent to the pericardial sac on its opposite side are warm and significantly perfused by blood. They may include the lung, diaphragm/liver, aorta, and others like the phrenic nerve—when unintentionally ablated will lead to paralysis of the diaphragm). Aside from damaging these organs unintentionally and possibly causing fistula creation (unwanted connections between the heart and adjacent organs—most described is with the esophagus), all of these organs are quite warm and perfused with blood. They will use up a degree of the ablation element's cooling power in areas of contact and decrease the potency of ablation where ablation is actually intended. A novel mitigation strategy would be to somehow insulate only one side or surface of the ablation element, while allowing the other side to remain exposed and available to contact tissue for intentional ablation. A major obstacle with this general concept is that the stronger the cryoablation energy, the more difficult it is to insulate it.

On the one hand, this type of device requires an incredibly potent cryoablation energy to penetrate and thoroughly ablate thick tissue like the left ventricle muscle. The various embodiments herein have a 3-dimensional focal ablation element at its tip that is powered by very cold cryofluid that has a vacuum-insulated conduit (catheter) through which the cryofluid is urged by pressure accumulation (at the cryofluid source). The cryofluid is pushed along the conduit via a dedicated inflow line to a cryoablation element near its tip. In order to achieve extreme cooling power at the ablation element, the element needs to remain extremely cold despite being warmed by the target tissue (and surrounding environment) as it draws in energy (heat). For extremely powerful systems, this may require much higher mass flow rates than typical commercial devices exhibit (over 120 standard liters per minute, but often as high as over 400 SLPM), and because the devices need to be small enough to be used in a minimally invasive environment, with progressively smaller conducting tubes, the cryofluid must flow at very high velocities, usually over 0.3 Mach, and sometimes at Mach speed (the speed of sound). One distinct advantage over a commercially available system that utilizes liquid nitrogen (or more specifically, near-critical point nitrogen) as the source cryofluid, is that extremely cold helium vapor is much less dense than liquid or NON-nitrogen and therefore can flow much more briskly and at much higher mass flow rates. The NON-nitrogen system requires very high driving pressures (~500 psi) in order to generate cryofluid mass flows of only ~70-80 SLPM. The high-pressure system represents a safety risk in the case of a catheter breach, and at least one related fatality has been reported. A much colder cryofluid system described herein based on liquid helium or neon can function at much lower pressures (~50-100 psi) yet produces mass flow rates of >300 SLPM. In both cases, the cryofluid flows into the ablation element via a dedicated inlet line and then out of the ablation element via a dedicated exhaust line. In such a fashion, the cryoablation element can be kept as cold (and potent) as possible while it contacts tissue, despite the tissue ‘resisting’ freezing by virtue of tissue thickness, perfusion with warm blood vessels, and the flowing warm blood pool inside the heart chamber in the case of cardiac ablation. The warm target tissue will be working to warm the ablation element during the ablation cycle, so a device that is designed to overcome any unwanted warming from adjacent tissues is highly desirable, regardless of cryogen temperature or driving pressure. For a potent ablation device that utilizes a very low temperature cryofluid at a high mass flow rate, insulation of any portion of the ablation element represents a significant challenge, since conventional insulating material (i.e. foams, plastic, other) would only be marginally effective. The most effective insulation is vacuum surrounding the cold elements, as is done in the conducting catheter described.

An additional type of effective insulation can also be accomplished by creating a threshold of space (lift) between the ablation element and adjacent tissues that could warm the ablation element on its ‘unintended’ or ‘non-working’ side or sides/areas. Importantly, this type of insulation device creates focal ablation element shielding in a specific way: it exposes only a lateral or side portion of the surface or circumference of an i.e. cylindrical (3-D) ablation element. This is especially important in epicardial ventricular ablation because of the angle of approach to the target tissue, which is essentially parallel. Meaning, that it would be more difficult to contact tissue with a small exposed (omnidirectional) catheter tip. This is in contradistinction to the usual type of adjustable shielding available with commercial cryoablation devices that are usually cylindrical—these usually consist of a coaxial sheath that surrounds the entire ablation element, effectively lengthening or shortening an otherwise omnidirectional cryoablation element. With an insulating chamber, the shorter the distance, the less effective the insulation is. One example of such a displacement device would be to use a balloon or pontoon, and to fill it with a gas that itself cannot be liquefied or frozen—a problem more unique to extremely low temperature (XLT) devices like those that might use XLT helium or neon. Gas within an insulation chamber that liquefies or freezes because of proximity to an XLT ablation element will become ‘ablative’ because the more compact nature of liquid or solid states is much more thermally conductive than in a ‘spread-out’ gas state. The only gasses that would not liquefy or freeze when exposed to temperatures associated with a liquid helium-driven system are helium and hydrogen. They are therefore low-thermal conductivity chambers because the space that is created will not significantly conduct the cooling energy associated with the ablation element to a significant extent and would be expected to prevent ablation of tissue on the opposing end of the displacement chamber. The helium gas may be added to the low thermal conductivity chamber slowly as the embedded gas contracts through a single channel or may be a flow-through insulation chamber with dedicated flow lines in and out of the insulation chamber while maintaining a set pressure or chamber volume. Another option would be an expandable open stent made of poorly conductive material. An important limitation regarding a displacement chamber/device when considering epicardial ablation is that you can only push the pericardium sac away from the heart surface to a limited extent. At a certain size limit, the heart will become compressed and will fail acutely and the result would be acute cardiogenic shock. While the maximum size limit for displacement depth (height) can be variable, in certain embodiments the height can range from about .5 cm to about 3 cm.

For a small-sized 3-D ablation element that would be suitable for focal epicardial ablation, there is another crucial problem to address. It would seem intuitive that a small ablation element suitable for focal ablation (most ablation catheters are in the 3-4 mm diameter range) should only require a similar-sized insulation chamber that can displace the ablation element yet is tall enough to create the distance required for effective insulation. Such a focal ablation element could be a small low-profile balloon, or a more rigid ablation chamber made of plastic or metal. The problem is that with a smaller ablation device and similar-sized insulation chamber, there will be a possibility for the combined catheter, ablation element and insulation apparatus to twist or flip around its long axis. If this occurs, then the ablation element may turn away from the target tissue or worse—it could flip 180 degrees and directly contact the pericardial sac and carry out the worst kind of (direct) unintended ablation. This would not be an issue with a larger surface area ablation element (and similar-sized insulation apparatus) and is more unique to smaller focal ablation elements in the epicardial space. The proposed solution is to control the shape of the insulating structure as it relates to the ablation element in a very specific way. It will still require a degree of ‘lift’ to separate the pericardial sac (and its heat) from the ablation element. An exemplary iteration may use a 1.5 cm height. This unexpected solution relates to the ‘real estate’ between the heart surface and pericardial sac and a physical impediment to rotation around the longitudinal axis. According to various embodiments, one of the insulation chamber surfaces that contacts either the heart or pericardial sac can be a substantially broader platform than the insulation chamber height, and broader or wider than the ablation element itself. With insulation chamber surface dimensions (length, width or circumference) that are notably larger than the height/depth of the chamber (like a sofa cushion), the chamber/cushion itself will occupy significant volume within the small pericardial sac, but without excessive depth that would cause cardiac compression. With such a design, it should be impossible for the combined (smaller) ablation element and insulation device to rotate away from the target or flip onto its side or all the way around (180 degrees). It will be restricted from axial rotation as a rule, and therefore assure that the ablation element will face and contact the heart and cannot be moved except along a plane that is in between and parallel to the surfaces of the heart and pericardial sac sheaves. This ensures that the ablation element remains not only ‘polar’ or restricted, but polar in the correct orientation. Note that the combined ablation and insulation device will be passed into the pericardial sac in a collapsed (minimally invasive) configuration, and once safely in the space between the heart and the heart sac, it is deployed to an open configuration. Its 3-D shape assures that the device is always deployed in a way that forces it to have one broad surface area contact the heart or contacting the pericardial sac. It will essentially be ‘jailed’ in this space and should only be able to move in a plane that is parallel to the surface of the heart (it can only slide between the sheets). The ablation element is secured to the surface of one of these two broad sides (or hemi-circumferences, in the case of a balloon or rounded structure). Correct orientation (ablation element contacting the heart while deployed) is assured by either or both of, markers on the catheter shaft (indicating on which side the ablation element lies) and/or electrical sensors on the ablation apparatus itself or the ‘ablation side’ of the inflatable cushion that confirm correct initial cardiac tissue contact. The latter can be temperature, magnetic, impedance, voltage, or piezoelectric contact force sensors, or any other type of sensor useful for localization of the ablation element. Sensors or markers alone would not assure continuous correct orientation or tissue contact during ablation or adjustment—electric or electromagnetic sensors usually temporarily lose function at cryogenic temperatures—and the broad platform insulation chamber is required to maintain correct orientation during a several-minute ablation and subsequent thaw period. For device embodiments that use an energy source like pulsed energy fields, such a platform creates a very useful polarity—the electrodes are assured of contact with the target tissue (the heart surface) and the insulation chamber is made to be minimally or non-electrically conductive. With such a configuration, the pulsed field energy that would typically be omnidirectional and conduct to non-target tissue either by contiguity or by arcing through gas (usually air) outside of the heart becomes polar or unidirectional and can be focused onto and into the target cardiac tissue. When the procedure is complete, the insulation chamber is deflated or collapsed, and the entire device removed via the remote pericardial access point as a single unit. Other device features might include, but are not limited to, elements embedded in the wall of the catheter/conduit that allow the catheter and ablation element/insulation chamber to be steerable in one or more dimensions.

An additional benefit of polar shielding using an inflatable chamber is that the force exerted between the sheets of pericardial sac and heart surface will tend to stabilize the beating heart in that particular target location, which facilitates correct positioning of the ablation element before it ‘sticks’ to the target by a process known as cryoadhesion when a cryothermal device is used. This effect could be further enhanced by adding a friction surface to the portion of the inflatable insulation chamber that contacts the surface of the heart and surrounds the ablation element. The friction surface would enhance stabilization of the heart segment that is compressed by the pontoon being sandwiched between the heart surface and pericardial sac.

Small-footprint (‘focal’) devices based on very cold cryofluid (XLT devices, or even warmer cryofluids like liquid nitrogen) that are used from inside of the heart also require attention to restriction of omnidirectionality for enhanced potency but do not require a large surface insulation cushion platform to prevent flipping/turning because the ventricle cavity is not defined by two closed apposed sheets or layers (like in epicardial ablation). In fact, such a bulky insulation chamber would be dangerous because of its volume displacement, which would likely cause significant obstruction of blood flow within a cardiac chamber, with the result of acute cardiogenic shock. In terms of insulation, this is also a special and unique design requirement because of how a cryoablation element works combined with the effect of immersing such an ablation element into an actively flowing (and constantly renewing) warm blood pool. The effective (warm) thermal mass of the entire body's flowing blood pool that passes through each heart chamber repetitively is massive. Ablation devices in general, and conventional/commercial cryoablation devices specifically, are omnidirectional. They absorb energy/heat from every exposed surface that can get cold. Typically, these devices comprise a catheter with inner tubing that conducts cold cryofluid to a special tip—the ablation element. While the catheter leading up to it requires some kind of insulation for prevention of unintended ablation, safety of handling and to prevent heat leak (cryofluid warming), the ablation element is purposely not insulated, so that it can get as cold as possible for the function of ablation of target tissue. These are usually cylinders (or cuboids/other) of a limited exposed axial length that can terminate bluntly or into a particular shape or even work by conducting the freezing through an endplate. But they all have one thing in common—they ablate circumferentially or omnidirectionally in their exposed surfaces. When an application requires a very high cooling flux, it usually requires most or all of the available cooling power to ablate target tissue. Consider a device powered by a very cold cryofluid comprising a catheter with a cylindrical ablation element at its leading end, that is passed via a peripheral blood vessel and into a heart chamber with intention to contact target tissue on the inside of the heart. Even if the ablation element contacts the heart muscle target, only a small percentage of its exposed surface area will actually contact the target tissue. The intended or possible contact area is often only i.e. 25-50% of the available surface area of the exposed ablation element, and more specifically, a side area as opposed to a tip may be the most convenient area of contact. The rest of the ablation element will be exposed to blood flowing in the heart chamber. This could comprise 50-75% (or more) of the exposed ablation element that is absorbing energy that does not contribute to ablation. The risk is not that the catheter will unintentionally ablate the blood. Rather, it is that the warm blood flowing around the catheter (and constantly being renewed with every heartbeat) is an extremely large source of heat. This heat will be absorbed by every available portion of the ablation element surface that is not contacting the target tissue. This process dramatically weakens the cooling power of the ablation element and will reduce its ability to deeply penetrate and thoroughly ablate the target tissue. These risks become more dramatic as the temperature of the ablation element decreases, because the forces at play will be driven by the temperature gradients. So, with a very cold (and especially with an XLT) cryofluid, its cooling power will want to draw energy from the warmest substrate in its vicinity—the renewable volumes of warm blood. This will diminish its cooling power delivery to the intended target (the contacted heart muscle). What is required is a way to configure or modify the ablation element so it becomes more unidirectional or polar than omnidirectional. If only certain portions of the ablation element are insulated (and thus energy absorption is eliminated in the exposed surface area not intended to be used for tissue contact), a more limited area of the ablation element is exposed, giving the ablation element a higher cooling flux (cooling per unit area). Ideally, the insulation will be so effective that minimal or no energy can be absorbed in any location except where contact with the target tissue is intended. For instance, this could comprise only 25-50% (or an even smaller window) of the lateral/side surface area of a cylinder or bubble-shaped ablation element. Importantly, such effective insulation elements must still allow the exposed portions of the ablation element protrude enough to contact tissue, i.e. the bulk of insulation cannot create a significant step-off that would create distance between the exposed ablation element and target tissue (and prevent contact). One way to overcome this is to use a low-profile balloon that protrudes or bulges slightly through a window created in a vacuum sheath assembly or using a rigid or semi-rigid ablation element designed to overcome any step-off.

Yet another innovation in creating polar cryoablation devices relates to heterogeneous areas of cooling power within the same cooling (ablation) element. Heat transfer between an ablation element and target tissue will be enhanced when all or the majority of the cryofluid mass flow is concentrated in one area of the interior of the ablation element, with more passive dispersal of cryofluid in the remaining interior space of the ablation element before it is exhausted. We describe devices that take advantage of this phenomenon by directing a highly concentrated jet of cryofluid and its associated cold atomic or molecular mass at a limited target area on the interior of the ablation element that is intended to contact tissue for ablation. The external surface area in this jet-cooling location then becomes ‘polar’ (an area of significantly higher heat transfer) with respect to the remaining surface area of the cooling element and is exemplified in an arrangement where the cooling jet is not merely a linear-directed extension of the cryofluid flowing in the ablation catheter inlet tube. Meaning, the cooling jet of cryofluid may be directed along any vector that causes it to concentrate cryofluid mass flow to a limited portion of the internal surface of a larger ablation element that is not directly ahead of the initial direction of cryofluid flow in the catheter. This augments the general concept of ablation element shielding—not only are portions of the ablation element inhibited from absorption of energy from adjacent tissue, but the unshielded (active) portion of the ablation element has enhanced cooling power by maximizing the cryofluid mass flow to the interior surface of the unshielded (active) area.

The best way to insulate any part of a device that works with a very low temperature cryofluid is by creating a vacuum jacket around the cold elements. In most cryoablation systems that use one, the vacuum jacket ends at the location where the ablation element comes out of the tip of the catheter. In the various implementations herein, an eccentric vacuum jacket is provided that is not simply a slidable cylindrical sheath that merely controls the axial length of a cylindrical ablation element. Regardless, a simple material sheath would not provide adequate insulation at these low temperatures of operation. Instead of the vacuum jacket ending at a plug or barrier through which the cooling tube extends inside the exposed (non-vacuum jacketed) ablation element, the novel catheter has a partial extension of the vacuum jacket, a hood or cradle that encases portions of the ablation element. This could be a hemi-jacket that surrounds the bottom 50%-75% of a cylindrical ablation element and may cover the cylinder end. The ablation element may also be of any shape that is not a cylinder, including lower profile designs that are relatively flatter. Many configurations of partial-coverage vacuum jackets can be imagined, with the common features that only a limited portion of the surface area of the ablation element is left exposed to contact tissue, usually a ‘side’ area and not along a complete circumference. Another important feature is that this is accomplished without the bulk of the eccentric vacuum jacket creating a recess that would prevent or inhibit the ablation surface from reaching or contacting tissue. In this fashion, only the exposed portion of the ablation element can draw energy from its surroundings or contacted tissue. This makes the exposed area of the ablation element much more potent than if the rest of it were exposed to the blood pool by increasing the ablation element's cooling flux substantially.

Another issue to contend with in terms of interference with maximal cooling is the transition area between the vacuum-insulated catheter and the ablation element. Often, this transition is a relatively long area where the vacuum seal occurs, essentially with a plug that allows one or more tubes to pass through it to conduct cold gas. This plug fluidically seals the end of the catheter tubing and allows vacuum to be applied to the inside of the catheter without vacuum loss. This end-plug can get very cold and become ‘ablative’ when using very low temperature cryofluid. The length and volume of material (and bonding) in the end plug allow for a durable seal between the plug and the inside of the outer (vacuum) catheter. Unfortunately, with very low temperature cryofluid material that usually gives reasonable insulation in this area is much less effective, and this transition zone where the vacuum jacket ends and the plug begins will become an ablation zone, depending on the distance between the cold line and the outermost surface of the plug, and the thermal conductivity of the plug material. Since minimally invasive ablation catheters need to be as small as possible for ease of navigation through peripheral blood vessels and the heart, there is a need to keep the catheter outer tube (vacuum jacket) as small a diameter as can be tolerable but still have effective ablation. To mitigate the end-plug area functioning as an unwanted ablation element, there are two solutions proposed. The first is to make the end-plug much shorter than usual, with welding and bonding techniques that will still be associated with a strong vacuum seal. Another solution is to extend the vacuum jacket up and over the end-plug or end-cap, to as to envelop the transition zone and insulate it sufficiently to prevent ablation in this area. Yet another technique would be to encase the transition zone with a balloon that is filled with a gas that will not freeze or liquify under XLT cryothermal conditions (usually helium gas). One limitation with an encasing balloon will be the size/volume required to mitigate ablation from this zone, which itself could either prevent good tissue contact with the adjacent ablation element or impede navigation within a complex cardiac chamber like the ventricle (characterized by irregular internal trabeculations that may limit navigation).

### BRIEF DESCRIPTION OF THE DRAWINGS

FIG. 1A is a perspective view of a known cryoablation catheter device.

FIG. 1B is an expanded perspective view of the ablation element of the device of FIG. 1A.

FIG. 1C is a perspective view of another known cryoablation catheter device with a slidable shield.

FIG. 1D is another perspective view of the device of FIG. 1C.

FIG. 2A is an ablation element of a cryoablation catheter device with an inflatable shield in its uninflated state, according to one embodiment.

FIG. 2B is a cross-sectional view of the device of FIG. 2A, according to one embodiment.

FIG. 2C is a perspective view of the device of FIG. 2A with the inflatable shield in its inflated state, according to one embodiment.

FIG. 2D is a perspective view of another device with an inflatable shield in its inflated state, according to one embodiment.

FIG. 2E is a perspective view of another device with a larger inflatable shield (in comparison to the shield of FIG. 2D) in its inflated state, according to a further embodiment.

FIG. 3A is a perspective view of a cryoablation catheter device with an uninflated pontoon shield, according to one embodiment.

FIG. 3B is an expanded perspective view of the device of FIG. 3A disposed between the pericardial sac and the surface of the heart, according to one embodiment.

FIG. 3C is another expanded perspective view of the device of FIG. 3B with the pontoon shield in its inflated state, according to one embodiment.

FIG. 4A is a perspective view of a cryoablation catheter device with dual shielding pontoons in their deflated state, according to another embodiment.

FIG. 4B is an enlarged perspective view of the cryoablation catheter device of FIG. 4A disposed between the pericardial sac and the surface of the heart with its dual shielding pontoons in their inflated state, according to one embodiment.

FIG. 5A is a perspective view of a hemispherically-shaped shielding pontoon for a cryoablation catheter device, according to one embodiment.

FIG. 5B is a perspective view of another hemispherically-shaped shielding pontoon with a groove defined therein, according to a further embodiment.

FIG. 6A is a perspective view of a cryoablation catheter device with a vacuum sled, according to one embodiment.

FIG. 6B is an enlarged perspective view of the distal end of the device of FIG. 6A, according to one embodiment.

FIG. 6C is an enlarged perspective view of the distal end of another device similar to the device of FIG. 6A with a shorter end cap, according to one embodiment.

FIG. 6D is an enlarged perspective view of the distal end of another device similar to the device of FIG. 6A having an ablation element that is an expandable balloon, according to one embodiment.

FIG. 6E is an enlarged perspective view of the distal end of a further device similar to the device of FIG. 6A in which the vacuum sled has extensions, according to one embodiment.

FIG. 7A is a perspective view of a distal end of a cryoablation catheter device with an ablation element that is an inflatable sphere, according to one embodiment.

FIG. 7B is a perspective view of a distal end of another device with another ablation element that is an inflatable sphere, according to another embodiment.

FIG. 7C is a perspective view of a distal end of yet another device with another ablation element that is an inflatable sphere, according to a further embodiment.

FIG. 8A is a perspective view of a shielding pontoon for a cryoablation catheter device, according to one embodiment.

FIG. 8B is a perspective view of the shielding pontoon of FIG. 8A with a cryoablation catheter device disposed therein, according to one embodiment.

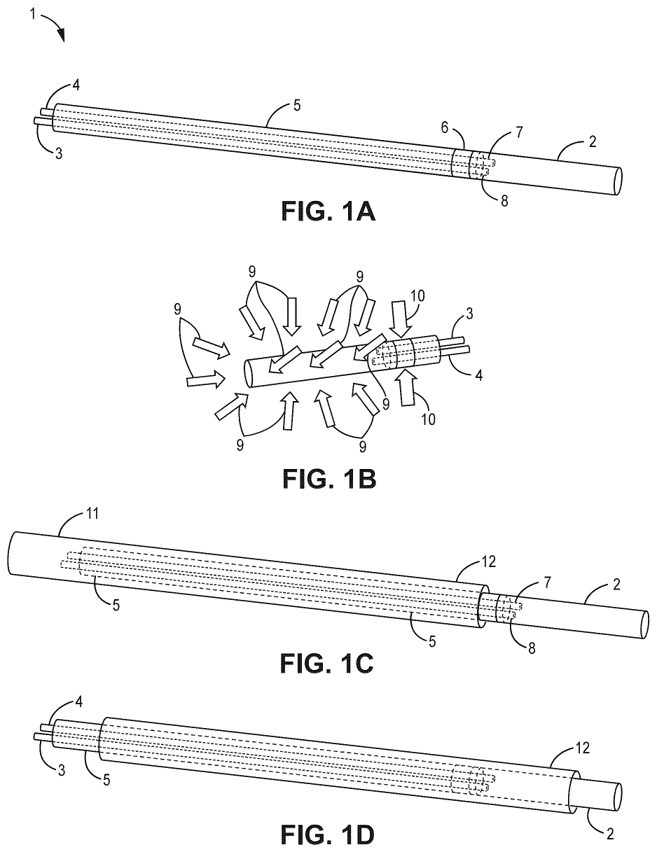

### DETAILED DESCRIPTION

The various embodiments herein relate to focal ablation devices that incorporate directed, non-omnidirectional ablation elements that direct ablation energy solely in the direction of the target tissue, thereby preventing ablative power from being wasted on other, unintended targets in the vicinity of the ablation elements. Certain embodiments are cryoablation catheter devices, but other forms of ablative energy are contemplated herein. In some implementations, the ablation devices can have shields that are positioned around a portion of the ablation elements such that the ablative energy is focused or directed solely toward the target area of the target tissue. For example, certain embodiments include partial or lateral surface or eccentric shielding of focal cryoablation catheters to convert them from omnidirectional energy absorption devices to more polar ones. Thus, the various implementations herein maximize the ablative power of the various devices herein in the face of significant factors that work to reduce the efficacy and efficiency of ablation. Of note, certain existing cryoablation devices include a slidable co-axial sheath that can reduce the axial length of the cryoablation element thereby reducing or increasing the length of an ablation footprint. However, these known devices are still omnidirectional in their exposed cryoablation elements. The various embodiments disclosed or contemplated herein address an important gap in the field because ablation devices—including cryoablation devices, for example—often need to ablate tissue on only on side of an ablation element, not circumferentially in all exposed surfaces. These non-participating areas significantly reduce the potency of ablation. If a co-axial sheath were used to encase almost all of a linear cylindrical (or cuboidal/other) shaped cryoablation element and just leave its very tip exposed, such a device would have to be flexed aggressively at close to 90-degrees in order to achieve some degree of tissue contact. The devices described below illustrate ways to insulate portions of side surfaces or expose only a limited amount of (non-tip) cryoablation element surface area in order.

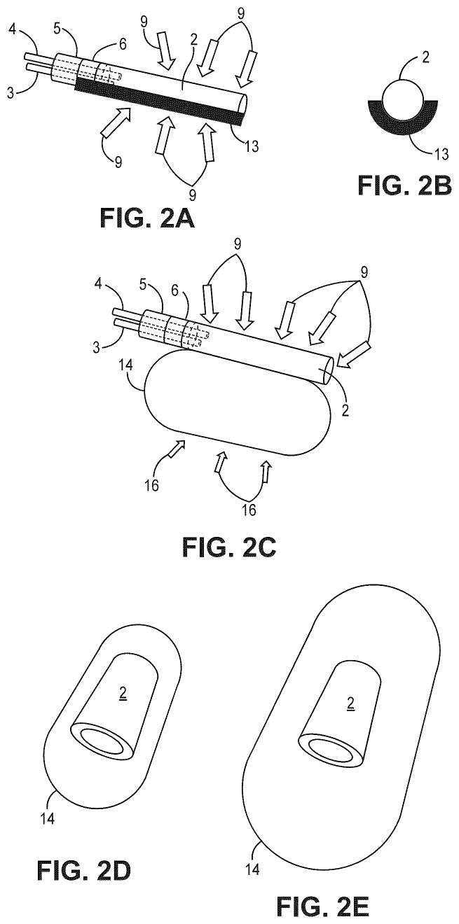

FIGS. 2A-2G illustrate some exemplary devices for shielding one side of a cylindrical ablation element 2, according to certain embodiments. Each of the devices herein has lines 3 and 4 that traverse an empty (“annular”) space that is fluidically sealed within outer tube or vacuum jacket 5 and intended to have a vacuum drawn on the annular space during operation, to insulate lines 3 and 4 while they are extremely cold and prevent heat leak and warming of the cryofluid. The vacuum also allows the outer surface of vacuum jacket 5 to be unaffected by the cryofluid and be safe for an operator to handle. Whereas in other cryoablation catheters, insulating material (foams, fabric, polymer) may be sufficient for insulation, in a catheter that utilizes very low temperature cryofluid (like helium at temperatures colder than −250 C) a vacuum is typically most effective. When a vacuum is used in this fashion, the cold lines 3 and 4 conducting the cryofluid must pass through an end-plug or end-cap 6 to ablation element 2. The function of end-plug 6 is to create the fluidic vacuum seal between the vacuum space and the ablation element 2.

FIG. 2A shows a device according to one embodiment having an uninflated shielding device 13 on only one aspect of ablation element 2, that is not co-axial. In this example, shield 13 is a non-inflated pontoon shield 13. As shown in FIG. 2A, the shield 13 in its uninflated state does not restrict energy absorption into ablation element 2 as depicted by energy absorption arrows 9 being unchanged in size or appearance on either side of ablation element 2. FIG. 2B is an en-face view from the distal end showing how shield 13 may be adhered to the bottom ~50% circumference of cylindrical ablation element 2. FIG. 2C depicts the device with the shield 13 inflated and expanded into a pontoon shield 14, that creates enough space underneath ablation element 2 using a low-thermal conductivity scheme (helium gas, in an exemplary device) that prevents energy/heat from being absorbed (tiny arrows 16) by the lower portion of ablation element 2. Note that the upper portion of non-shielded ablation element 2 can absorb heat/energy as usual, and in fact can do so more effectively because its bottom half of cylinder surface area cannot contact a source of warmth/energy. FIG. 2D shows a device implementation with a shielding pontoon 14 having a diameter that is at least 1.5× the diameter of ablation element 2. According to certain embodiments, the diameter being at least 1.5× the diameter of the ablation element reduces the risk of twisting or torsion about the elongate axis of the device during use. Note than in the case of a single pontoon that is larger than the ablation element, the upper hemi-circumference will be equal to (pi×(diameter/2)). This equates to the hemi-circumference being slight greater than 1.5× of the pontoon diameter. FIG. 2E shows that, in some embodiments, the torsion risk may diminish further as shielding pontoon 14 diameter is progressively larger in comparison with ablation element 2. Twisting/torsion 15 could result in the unshielded portion of ablation element 2 turning out of position and inadvertently contacting non-target areas. This is a particular issue for epicardial ventricular ablation, as the ablation device is navigated in a limited space called the ‘pericardial space’. Epicardial ablation procedures are usually done without direct or indirect vision, rather guided by electrical or magnetic signals that do not predict behavior like twisting/torsion, making this a very important risk.

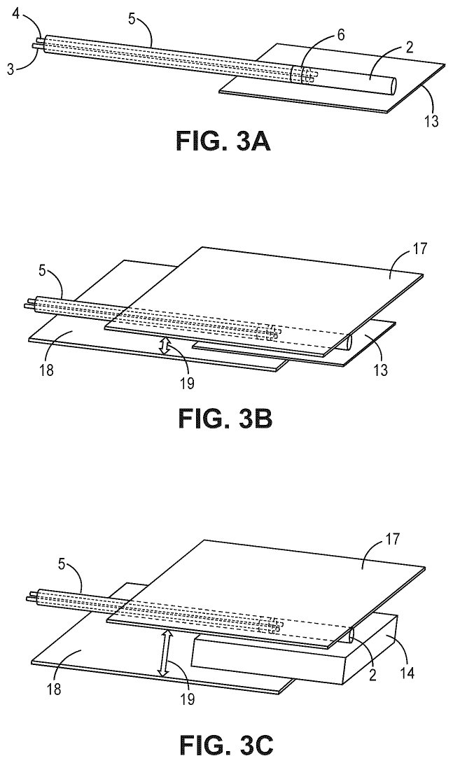

FIGS. 3A-C represent another solution to twisting/torsion of a cryoablation catheter with an ablation element 2 at its tip, maintaining its new polarity (restriction of omnidirectional action) in the correct orientation. FIG. 3A shows another example of a pontoon shield 13, un-inflated. This configuration would not inhibit energy absorption into the bottom portion of ablation element 2. Movement and navigation of a catheter device for epicardial (external) ventricular ablation is like moving along the space between two layers 17 and 18 (facilitated by a very thin layer of lubricating pericardial fluid) in FIG. 3B. The two layers can be pushed apart somewhat by the catheter device. In the example of FIGS. 3B and 3C, the surface of the heart is represented by layer 17 and the adjacent pericardial sac is represented by layer 18. Immediately on the other side of pericardium 18 (not shown) are organs that comprise significant sources of heat, like the aorta, diaphragm/liver. In this example, the space 19 in between the two layers 17, 18 is pushed apart by inflation of a broad quadrangular-shaped shielding pontoon 14, whose height/depth must be limited in order to not compress the heart 17 excessively (this would compromise heart function). In various implementations, the height inflated shielding pontoon 14 is less than around 3 cm. Alternatively, the height of the space 19 can be any amount that does not result in excessive heart compression. In this particular implementation, the top area of shielding pontoon 14 forms a platform upon which the distal portion of ablation catheter 5 and ablation element 2 rest. In this example, the shortest dimension of the platform is at least 1.5× the height of inflated pontoon shield 4. This creates a relatively broad, flat inflated cushion 14 that is tall/deep enough to restrict energy absorption in the bottom ~50% surface area of ablation element 2. This configuration makes the ablation element 2 more unidirectional than omnidirectional, and the unique shape of the inflated shielding pontoon 14 restricts movement between layers 17 and 18 so that (now polar) ablation element 2 always faces its intended target surface 17 (the heart). In the example of the ablation element 2 comprising a mechanism to deliver a pulsed electrical field, also an inherently omnidirectional source of energy, the shielding pontoon works to electrically insulate all but a limited direction of energy delivery. More specifically, the electrical pulses that cause irreversible electroporation in the polar aspect of contacted tissue are more potent, because the shielding pontoon is highly resistant to conduction of electricity in the areas where energy dispersion would be useless, wasteful or unintentionally injurious. Importantly, simply covering one side of a pulsed electrical field device's electrodes would have a limited effect on restriction of energy dispersion on that side of the device. The large size and volume of the inflatable or expandable shielding pontoon creates a large zone of high electrical resistance that minimizes or eliminates energy dispersion (including electrical arcing through air to nearby conductive tissue) except in the non-shielded, polar area of intended ablation.

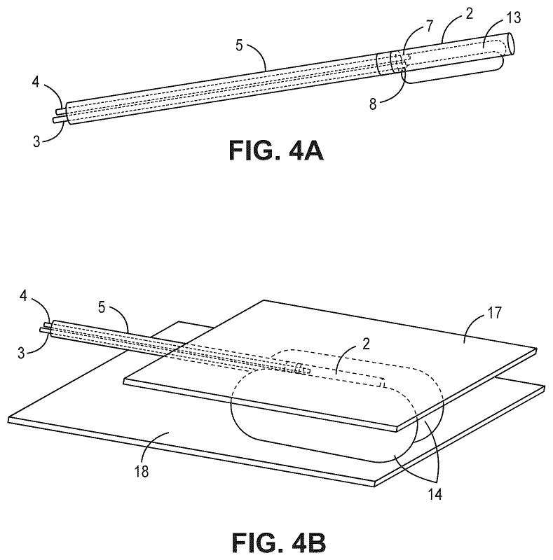

In the example in FIGS. 4A and 4B, rather than a single cylindrical shielding pontoon, the device has double shielding pontoons 13 in order to create a broader platform for anchoring and shielding the ablation catheter compared with a single shielding (cylindrical) pontoon. In FIG. 4A, the device has deflated pontoons 13 that would not be expected to inhibit energy absorption below ablation element 13 if it were in proximity to a heat source. In contrast, FIG. 4B shows the inflated double shielding pontoons 14 side by side, between two heat sources 17 (the heart epicardial surface) and 18 (the pericardial sac and its adjacent warm organs). The significant displacement of layer 18 makes energy absorption by the lower portion of ablation element 2 very limited. Further, in this example, one can see that ablation element 2 is nestled significantly in between the two shielding pontoons 14, such that less than 50% of the upper circumference and surface area of the cylindrical ablation element 2 is exposed and available for energy absorption. This creates a more ideal situation wherein the exposed area on ablation element 2 is the only portion that can absorb energy because it is that only portion that can contact warm tissue (layer 17—the heart surface). Its cooling flux (energy absorption per unit area) is increased.

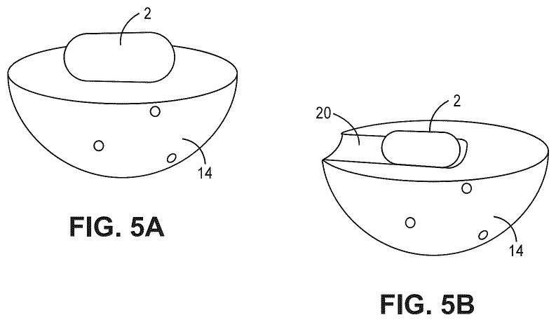

FIGS. 5A and 5B show that a number of inflatable shielding pontoon designs can be imagined. In FIG. 5A, an inflated hemispheric shielding pontoon 14 creates significant distance underneath ablation element 2. FIG. 5B shows an alternative device in which a groove 20 is engineered into this custom shielding pontoon 14 such that the ablation element 2 rests in this recess, so that only a very limited portion of its upper surface area remains exposed and available for energy absorption (cryablation).

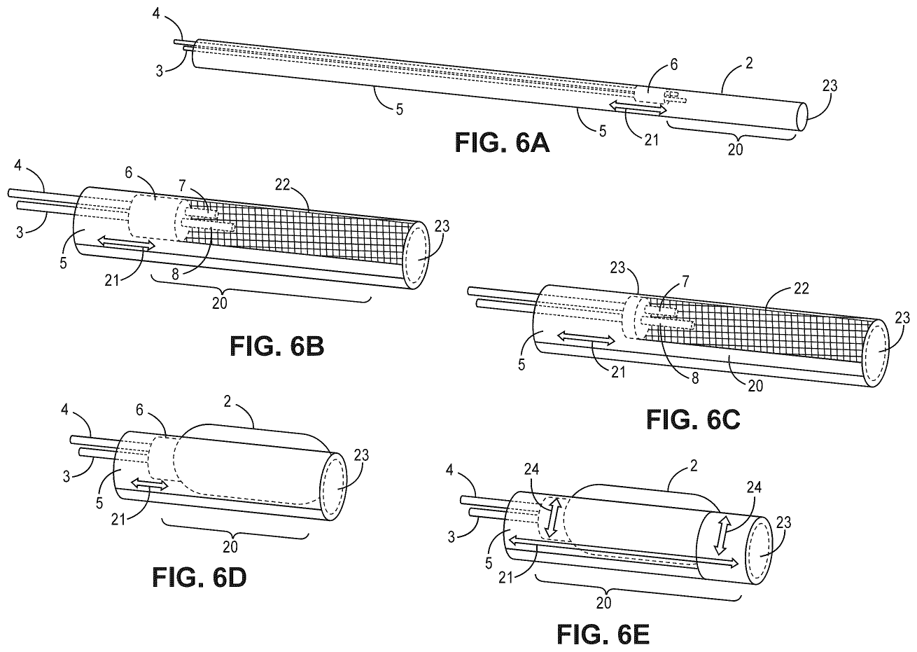

FIGS. 6A-6E show examples of cryoablation element side-portion shielding that do not utilize inflatable pontoons, but rather, vacuum sleds or cradles. According to certain embodiments, these devices with vacuum sleds as described in further detail below can be well suited for endocardial ablation within a cardiac chamber, or for videoscopic solid tumor cryoablation in a created space (i.e. laparoscopy, thoracoscopy). FIG. 6A shows a version of cryoablation catheter 1, which has a fluidic extension 20 (or “vacuum sled”) of vacuum jacket 5 that is in communication with the applied vacuum as evidenced by arrow 21. Vacuum sled 20 terminates in sled end 23, which itself may also have an annular space and communication with the applied vacuum. Within the vacuum sled 20 lies cryoablation element 2. Note that the sled 20 encases the bottom 50% or more of cryoablation element 2 and provides superior inhibition of energy absorption in these surfaces. Only the top ~50% of cylindrical ablation element 2 can absorb energy. As such, when it comes into contact with target tissue, the cooling flux that would normally be applicable to the entire surface area of cryoablation element cylinder 2 only applies to half of the cylinder's surface area, thereby increasing the effective cooling flux (cooling power per unit area) in the tissue contact area. The shielded portions of ablation element 2 cannot absorb heat from the surrounding blood pool and do not have an opportunity to warm the cryofluid mass that works to keep cryoablation element 2 as cold as possible. In certain alternative embodiments, the device can also have wires (not shown) embedded axially within the wall of the vacuum jacket 5 to mediate steering of the elongate vacuum jacket leading end. FIG. 6B is an expanded view of the distal end of this catheter, with the cryoablation element removed. Vacuum sled 20 can be better seen now as an extension of vacuum jacket 5, through whose annular space cryofluid inlet and outlet tubes 3 and 4 pass and to which vacuum is applied. Note that end-plug 6 does not fill the entire annular space of vacuum jacket 5, but instead allows a corridor 8 to remain open for fluidic communication of vacuum between vacuum jacket 5 and vacuum sled 20. As noted above, in this illustration, vacuum sled 20 is empty, and its unfilled interior 22 will have a cryoablation element implanted (not shown) that seals to end plug 6 and receives extensions of cryofluid tubes 7 and 8. Because end-plug 6 does not have vacuum insulation itself, with progressively colder cryofluids, it is likely to become an ablation area in and of itself.

Hence, FIG. 6C shows an alternative embodiment having a much shorter vacuum end-cap 23, as compared with end-plug 6. Techniques to assure a fluidic seal despite a shorter length plug are employed, including chemical and ultraviolet welding and material matching. In such a device, the end-cap may be much shorter, such as 6 mm or smaller in axial length, to decrease unintended ablation in this area.

While cryoablation element 2 may be comprised of metal or plastic and be of any shape to which a vacuum sled can mirror for shielding, FIG. 6D shows a further exemplary embodiment in which cryoablation element 2 is a low-profile balloon that bulges slightly so that its upper 50% circumference is available for energy absorption by contacting target tissue.

Further, FIG. 6E shows another device implementation having a more extensive vacuum sled 20 with extensions covering end-plug 6 and terminal sled end 23, with fluidic vacuum communication with catheter vacuum jacket 5 depicted by communicating arrows 21 and 24. In this model, the sled 20 also covers more of the balloon element 2 with only the top 20-30% exposed and available for energy absorption. Because most of the cryoablation element 2 cannot absorb energy/heat, all of the cryofluid mass that passes through it can only have an effect in the ‘open side window’ where it can contact tissue. This makes the contact area of the cryoablation element significantly more powerful than usual.

Further alternative designs include those in which vacuum sled 20 is a modular add-on to catheter ablation unit 1 and derives its vacuum from a source that does not directly communicate with the annular space of vacuum jacket 5 through a corridor 8, but rather from a dedicated vacuum line originating in the console connected to the vacuum sled 20 (not shown).

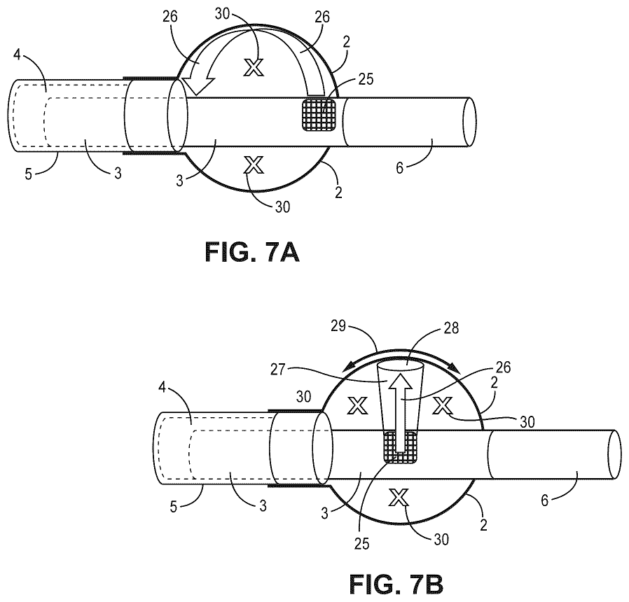

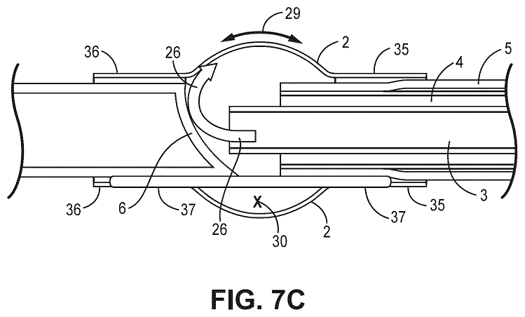

FIGS. 7A-7C show examples of directed jet-cooling within a larger ablation element. While an expandable shielding pontoon or apparatus 14 is not shown in these embodiments, such a component can be incorporated in these iterations, with the ablation element 2 joined to one surface or embedded within it. The cooling inlet tube 3 may be adjacent to or nested concentrically or coaxially inside of exhaust outlet tube 4, both of which are insulated by annular vacuum space between exhaust tube 4 and external vacuum jacket 5. Source of vacuum to the annular space is not shown. Cold inlet line 3 delivers cryofluid to the interior of ablation element 2, which in these exemplary embodiments is a sphere that can be an inflatable sphere 2 that expands from a previously collapsed configuration (not shown). Vacuum jacket 5 and cold inlet line 3 are bonded to short neck extensions 35, 36 (as shown in the embodiment of FIG. 7C) of sphere 2 to create a fluidic seal to sphere 2. In this example, the proximal neck 35 is larger than the distal neck 36 to accommodate the size difference between vacuum jacket 5 and inlet tube extension 3. End plug 6 occludes cold inlet line 3 and distal neck extension 36 so that no cryofluid can exit from the end of inlet line 3 or the ablation sphere 2 distal neck extension 36. Instead, in the device embodiment of FIG. 7A, an opening 25 in the distal aspect of cold inlet line 3, centered along its top (12 o'clock) portion, allows cryofluid that is urged under pressure from a proximal location and source (not shown) to exit the inlet line and curl 26 along a limited portion of the 3D perimeter of sphere 2 so that only a limited portion of the interior of the sphere is in contact with the initial bolus of ejected cryofluid, thereby enhancing heat transfer (ablation power) along the surface of this location. This initial bolus of ejected cryofluid will be more ‘concentrated’ (and therefore more potent) since the cryofluid atoms or molecules will not yet have had a full opportunity to expand into the available space in the ablation element. While the cryofluid ultimately exits via exhaust line 4 to leave ablation sphere 2 as inlet line 3 continuously supplies new and very cold cryofluid to the enhanced heat transfer zone, other portions of the interior of the sphere 30 have relative stagnation of cryofluid flow despite some lesser degree of heat exchange. This creates a favorable situation for the coldest portion 26 of the periphery of sphere 2, since these stagnant areas 30 will act as functional insulators. FIG. 7B represents an alternate implementation in which the opening 25 is located in the center of the sphere 2. This orientation allows for a ~90 degree turn in the cryofluid flow vector originating in inlet line 3, so that the exit jet 26 of cryofluid is ejected towards the 12 o'clock position of ablation sphere 2. Note that in this example, the ejection jet 26 results in a conical dispersion 27 of cryofluid that strikes the periphery of ablation sphere 2 at location 28. The mass of cryofluid (with maximal cooling power and heat exchange) radiates from location 28 along a limited 3D surface area 29 along the sphere 2 surface area and represents the most potent cooling area of spherical ablation element 2, rendering it polar. Again seen are relative flow voids 30 that are functional insulators and are exposed to a lower cryofluid atomic or molecular mass flow. Flow is continuous in the ablation sphere 2, with cryofluid ejected via opening 25, creating a higher heat-transfer zone 29 compared with other areas of sphere 2 and exhausting out of sphere 2 via co-axial or concentric exhaust line 4.

Another exemplary device embodiment that can create polarity and asymmetric enhanced cooling within an ablation chamber is shown in FIG. 7C, which includes deflecting the ejection jet 26 from the end of a non-plugged inlet tube 3 against an angled or curved plug 6 that directs the coldest and most concentrated cryofluid preferentially towards one area of the ablation chamber (i.e. the upper half of a spherical ablation element) that becomes a high-heat transfer zone 29, while creating a lower thermal transfer zone 30 away from the deflected jet (i.e the lower half of a spherical ablation element). In this iteration, because the cryofluid flow comes out of the tip of inlet line 3, the ablation element or sphere 2 may be supported by a spine or a bridge 37 that spans the vacuum jacket 5 or proximal exhaust tube 4 to a distal tube or fixation feature 6 that plugs ablation sphere 2 at its distal neck area 36. The spine or bridge 37 may be comprised of a stiff plastic or metal like stainless steel to function as a rigid support member. The inlet line 3 may be attached to the spine or bridge 37 or to the distal neck plug 6 to stabilize its position and prevent retraction when the inlet line 3 gets cold (not shown). It is expected that the proximal 35 and distal 36 neck extensions of ablation element or sphere 2 are locations for direct or indirect fixation of the ablation sphere 2 to the insulation chamber 14 (not shown).

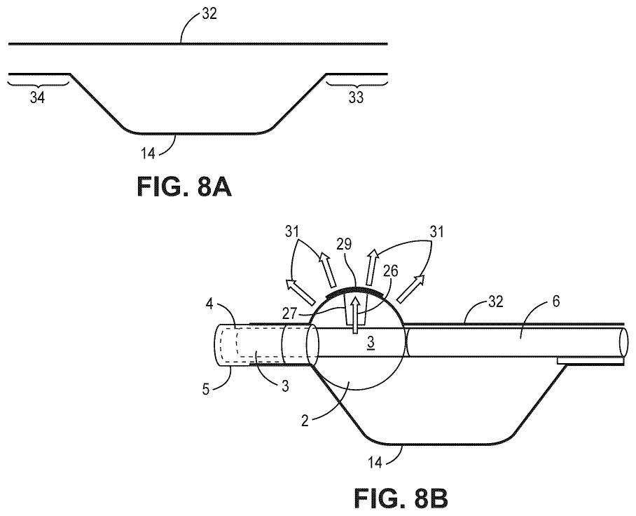

FIGS. 8A-8B show another example of asymmetric shielding or insulation of an ablation element, according to a further embodiment. More specifically, what is shown is a balloon-in-balloon configuration with asymmetric positioning such that only a limited portion of the surface area of the ablation element can come into contact with a limited portion of the wall of the insulation/shielding chamber, rendering an otherwise omnidirectional ablation element into a polar ablation element. FIG. 8A shows an eccentrically shaped shielding pontoon 14. Technically, this shape is called a “hemi-balloon” and is a specialty balloon shape that is commercially available. It has one side that is relatively flat 32 and an opposite side that inflates/bulges, and distal 33 and proximal 34 neck zones. Alternatively, a standard pontoon or balloon may be rendered asymmetrical by offsetting the locations of the neck portions of the shielding pontoon to a non-coaxial location. FIG. 8B shows an ablation device that has been inserted into the interior of hemi-balloon pontoon 14, which locates the ablation device asymmetrically within the pontoon 14, and more specifically, closer to its flat surface 32. It is sealed by bonding in neck zones 33 and 34. Note again, the components of the ablation element include cryofluid inlet line 3 that resides coaxially within the exhaust tube 4, both of which are contained within vacuum jacket 5 beyond an annular vacuum space for insulation. Cryofluid inlet line 3 extends within ablation element 2 (shown here as spherical, but could be any other useful shape), and is plugged distally by end plug 6 that is distinct from the annular plug (not labeled) that seals the exhaust tube 4 to vacuum jacket 5. This extended inlet tube 3 and plug 6 seal into the distal neck zone 33, and cryofluid exits the inlet tube 3 at a location inside ablation element 2 via an opening (not shown). In this example, ablation element 2 is initially collapsed (not shown), and cryofluid enters and pressurizes inflatable ablation element 2. Because of the proximity between a limited portion of the external wall of ablation element 2 and the internal wall of flat surface 32 of hemi-balloon 14, flat surface 32 is displaced outwards 31. This displacement is directed towards the target tissue for ablation so that limited flat surface portion 29 (that is directly apposed to the external surface of a limited portion of inflated ablation element 2) becomes a high heat transfer zone when it contacts tissue for ablation. This effect is amplified when jet cooling is utilized as described in FIGS. 7A-C. It is shown in FIG. 8B as a directional cooling jet of cryofluid 26 causing a non-annular or non-circumferential dispersion 27 of cryofluid that is directed towards high heat transfer zone 29 that is a result of displacement or tenting of flat surface 32 due to inflation of ablation element 2 that was in very close proximity to its internal surface. Further, the portions of inflated ablation element 2 that are not in contact with flat wall 32 or any wall or boundary of hemi-pontoon 14 are therefore shielded or insulated by the helium gas that is used to inflate hemi-pontoon 14, and the space created around the non-contacted surface areas of ablation element 2 cannot absorb heat from this low-thermal conductivity chamber that is kept filled with tepid helium gas via its own dedicated line (not shown). This effectively increases the polarity and the cooling flux of ablation element 2 in areas where it can indirectly draw energy out of target tissue via conduction through a limited amount of intervening hemi-balloon 14 material at area 29.

It is understood that all the examples may also use other types of non-cryogenic thermal ablation energy like radiofrequency, ultrasound, microwave as well as non-thermal energy sources like pulsed electrical energy. In these examples, cold inlet tube 3 and exhaust tube 4 are replaced with electrical components and annular space vacuum is not necessary. The ablation element 2 would be positioned against target tissue and the shielding pontoon would restrict dispersion of either heat or electricity (i.e., pulsed electrical fields) anywhere except into the target tissue (i.e. adjacent to flat surface 32 in FIG. 8), rendering these energy sources polar and more potent in the target area, whereas without the shielding pontoon they would function omnidirectionally, and sacrifice potency in the targeted area.

It is also understood that while the various ablation element embodiments described herein are generally round or cylindrical, the various shielding elements and structures disclosed or contemplated herein could also be used in combination with various ablation elements with flat or substantially flat surfaces as well.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

Although the various embodiments have been described with reference to preferred implementations, persons skilled in the art will recognize that changes may be made in form and detail without departing from the spirit and scope thereof.

What is claimed is:

1. An ablation device comprising:
a) an elongate body;
b) an omnidirectional ablation element disposed at a distal end of the elongate body; and
c) an ablation shielding structure attached to a first side of the omnidirectional ablation element, the ablation shielding structure comprising a low energy conductivity structure, wherein the ablation shielding structure is configured to resist energy conduction only on the first side of the omnidirectional ablation element, thereby increasing ablation potency on a second side opposite the first side.

2. The ablation device of claim 1, wherein the elongate body comprises a vacuum-insulated space, wherein the omnidirectional ablation element comprises a cryofluid disposed within the ablation element, wherein the cryofluid has a temperature of about 77 K or colder.

3. The ablation device of claim 1, wherein the omnidirectional ablation element comprises pulsed electrical field energy.

4. The ablation device of claim 1, wherein the ablation shielding structure is disposed around at least 50 percent of an exposed surface area of the omnidirectional ablation element.

5. The ablation device of claim 1, wherein of the ablation shielding structure comprises at least one inflatable pontoon.

6. The ablation device of claim 1, wherein the ablation shielding structure comprises a friction surface on a side of the ablation shielding structure facing the ablation element, wherein the friction surface is configured to stabilize the target tissue until cryoadhesion occurs.

7. The device of claim 1, wherein the ablation shielding structure has maximum height of between about 0.5 cm and about 3 cm.

8. The device of claim 1, wherein the ablation shielding structure comprises a shape and dimensions that are suitable for navigation and manipulation within a space between two adjacent displaceable layers and forms a platform whose shape restricts it from rotating or flipping around the long axis of the device.

9. The device of claim 1, wherein the ablation shielding structure is configured to create space between a left ventricle and pericardial sac to inhibit energy absorption from structures immediately adjacent to an outer surface of the pericardial sac.

10. The device of claim 1, wherein the elongate body comprises a first vacuum-insulated space, wherein the ablation shielding structure comprises a low-profile cradle of the elongate body comprising a second vacuum-insulated space, wherein the cradle surrounds a portion of the ablation element and leaves only a limited surface area of the ablation element unshielded.

11. The device of claim 1, wherein the ablation shielding structure is a balloon that surrounds portions of the ablation element and leaves only a limited portion or side of the ablation element exposed and available for ablation.

12. The device of claim 1, wherein a portion of the ablation element that remains exposed is a side or lateral portion.

13. The device of claim 1, wherein the ablation shielding structure is configured to maximize ablation power of the exposed ablation element by restricting energy absorption or delivery in other surfaces of the ablation element.

14. The device of claim 1, wherein the elongate body comprises embedded hardware in its outer portion to mediate steerability of the elongate body in at least one direction.

15. The device of claim 1, further comprising sensors to detect at least one of temperature, electrical impedance or voltage, position and contact force.

16. An ablation device comprising:
a) an elongate body comprising a lumen defined within the elongate body, wherein the lumen is configured to have a vacuum applied thereto;
b) an ablation element disposed at a distal end of the elongate body;
c) a conduit disposed within the lumen and in fluidic communication with the ablation element, wherein the conduit is configured to allow ablation fluid to pass through the conduit; and
d) an ablation shielding structure attached to the ablation element, wherein the ablation shielding structure is configured to block ablative conduction to or from a limited portion of a surface of the ablation element,
wherein the vacuum insulates the conduit within the lumen, and
wherein the ablation element is configured to direct the ablation fluid to a limited surface area of the ablation element that is rendered into a higher heat transfer area than other portions of the ablation element.

17. The device of claim 16, wherein the ablation shielding structure comprises a low-thermal conductivity chamber.

18. The device of claim 16, wherein the higher heat transfer area is not directly ahead of the flow of the ablation fluid in the conduit.

19. The device of claim 16, wherein the ablation element comprises a collapsed configuration and an expanded configuration.

20. The device of claim 16, wherein the ablation shielding structure comprises an inflatable or expandable low thermal conductivity chamber, wherein the ablation element is disposed within the low thermal conductivity chamber such that the limited surface area is disposed within close proximity or contact with a limited portion of an interior boundary of the expandable low thermal conductivity chamber.

21. The device of claim 16, wherein positioning of the ablation element inside the low thermal conductivity chamber causes displacement of the adjacent walls of the ablation element and the low thermal conductivity chamber into one another when inflated.
